(19) United States
(12) Patent Application Publication
(10) Pub. No.: US 2026/0159495 A1
(43) Pub. Date: Jun. 11, 2026

(54) METHOD FOR PROCESSING A LACTIDE FEED

(71) Applicant: Purac Biochem B.V., Gorinchem (NL)

(72) Inventors: Pornchai SAELIM, Gorinchem (NL); Rens VENEMAN, Gorinchem (NL); Gerrit GOBIUS DU SART, Gorinchem (NL); Hans DE VRIES, Gorinchem (NL); Carlos Alberto Gonzales RUGERIO, Gorinchem (NL)

(21) Appl. No.: 19/463,315

(22) Filed: Jan. 29, 2026

Related U.S. Application Data
(63) Continuation of application No. PCT/EP2024/071992, filed on Aug. 2, 2024.

(30) Foreign Application Priority Data
Aug. 3, 2023 (EP) 23189530.1

(51) Int. Cl. C07D 319/12

(52) U.S. Cl. CPC C07D 319/12

(57) ABSTRACT

A method for processing a lactide feed to form lactide streams with high configurational and chemical purity, comprising the steps of
subjecting a feed comprising L-lactide, meso-lactide, water, lactic acid, and optional further components to a first separation step, resulting in a stream comprising lactic acid and water and one or more crude lactide streams,
subjecting a crude lactide stream comprising L-lactide and meso-lactide resulting from the first separation step to a first crystallisation step, resulting in a purified L-lactide stream and a remnant stream comprising L-lactide and meso-lactide,
subjecting the remnant stream comprising L-lactide and meso-lactide resulting from the first crystallisation step to a second separation step, resulting in at least a second L-lactide stream and a meso-lactide stream, and
subjecting the meso-lactide stream resulting from the second separation step to a second crystallisation step, resulting in a purified meso-lactide stream and a further remnant stream.

### CROSS-REFERENCE TO RELATED APPLICATION

The present application is a continuation of International Application No. PCT/EP 2024/071992, filed Aug. 2, 2024, which claims priority to European Patent Application No. 23189530.1, filed Aug. 3, 2023, both of which are hereby incorporated by reference herein in their entireties.

### TECHNICAL FIELD OF THE INVENTION

The present invention pertains to a method for processing a lactide feed. The present invention also pertains to a method for producing a polylactide.

### BACKGROUND OF THE INVENTION

Polylactide, also known as poly(lactic acid) or PLA, is a polymer that finds application in a variety of fields, ranging from medical applications to packaging and disposable tableware. Polylactide is a polymer derived from lactic acid. Lactic acid is a chiral molecule and so exists as either(S)-or (R)-lactic acid, with (S)-lactic acid being the predominant form in nature. Commercially available polylactides generally contain a large proportion of (S)-lactic acid units and are usually obtained by ring-opening polymerization of (predominantly) L-lactide, a dimer of (S)-lactic acid.

Lactides are commonly synthesized by oligomerizing lactic acid to form lactic acid oligomers via a polycondensation reaction. These oligomers are then depolymerized to form a crude lactide comprising L-lactide ((S, S)-lactide), D-lactide ((R, R)-lactide), and meso-lactide ((S, R)-lactide). When the oligomers are synthesized from (S)-lactic acid, the resulting oligomer will contain mostly (S)-lactic acid units. Depolymerization of such an oligomer will, in turn, lead to a crude lactide in which L-lactide is the predominant stereoisomer. D-lactide and meso-lactide result from the presence of (R)-lactic acid, which may either be present in the original feed, or which may be formed through racemisation.

L-lactide, D-lactide, and meso-lactide can be sent to a polymerization reactor in various ratios. The ratio in which the lactides are sent to the polymerization reactor determines the ratio of (S)-lactic acid units to (R)-lactic acid units in the polymer, as each L-lactide molecule contributes two (S)-lactic acid units to the polymer, each D-lactide molecule contributes two (R)-lactic acid units to the polymer, and each meso-lactide molecule contributes one(S)-lactic acid unit and one (R)-lactic acid unit to the polymer. The ratio of (S)-lactic acid units to (R)-lactic acid units in the polymer is important, because it determines, for a large part, the physical properties of the polylactide. A polylactide comprising mainly (S)-lactic units and, say, 10% of (R)-lactic acid units will, for example, be less crystalline than a polylactide comprising only (S)-lactic acid units.

However, the ratio of (S)-lactic acid units to (R)-lactic acid units in the polymer is not the only factor that contributes to the physical properties of the polylactide. Another important factor is the presence or absence of impurities, such as hydroxyl-containing impurities, in the lactide stream that is sent to the polymerization reactor. Hydroxyl-containing impurities, if present in the lactide stream that is polymerized, reduce the average molecular weight of the resulting polylactide. Impurities may also affect the colour of the polylactide, its thermal stability and its suitability for food contact applications.

Many methods for processing lactide feeds have been disclosed in the art. For example WO2014180836 describes a process for the recovery and production of meso-lactide from a crude lactide containing stream, which comprises the steps of:
a. Subjecting a starting crude lactide stream to a first distillation step to obtain a top stream mainly containing meso-lactide, a bottom stream and a side stream mainly containing L-lactide and meso-lactide;
b. Recovering the side stream and subjecting said stream to a melt crystallisation step to obtain a first purified stream mainly containing L-lactide and a drain stream mainly containing meso-lactide and L-lactide;
c. Recovering the top stream issued from step (a) and the drain stream issued from step (b); d. Subjecting the top stream and the drain stream to a second distillation step to obtain a second purified stream containing L-lactide and meso-lactide.

WO2014/139730 describes a process for purifying lactide comprising the steps of
(a) separating a lactide-containing stream into one or more lactide-containing vapour fractions and one or more lactide-containing liquid fractions;
(b) condensing a lactide-containing vapour fraction as obtained in step (a) to obtain a lactide condensate;
(c) subjecting at least part of the lactide containing condensate as obtained in step (b) to melt crystallisation to obtain a purified lactide containing stream and a residue stream; and
(d) recovering the purified lactide-containing stream as obtained in step (c).

However, given the increased interest in polylactide for many applications, there is a need in the art for a method for processing lactide feeds which, on the one hand, results in the formation of lactide streams with a high configurational and chemical purity, while on the other, it can be performed in an efficient and cost-effective manner. Here, a high configurational purity refers to a high content of the desired stereoisomer. A high chemical purity refers to a low content of non-lactide contaminants. The present invention provides such a process.

### SUMMARY OF THE INVENTION

The invention pertains to a method for processing a lactide feed, comprising the steps of
subjecting a feed comprising L-lactide, meso-lactide, water, lactic acid, and optional further components to a first separation step, the first separation step resulting in the formation of a stream comprising lactic acid and water and one or more crude lactide streams,
subjecting at least a portion of a crude lactide stream comprising L-lactide and meso-lactide resulting from the first separation step to a first crystallisation step, the first crystallisation step resulting in a purified L-lactide stream and a remnant stream comprising L-lactide and meso-lactide,
subjecting the remnant stream comprising L-lactide and meso-lactide resulting from the first crystallisation step to a second separation step, the second separation step resulting in at least a second L-lactide stream and a meso-lactide stream,
subjecting at least a portion of the meso-lactide stream resulting from the second separation step to a second crystallisation step, resulting in the formation of a purified meso-lactide stream and a further remnant stream.

It has been found that the specific step sequence of the present invention results in the formation of an L-lactide stream and a meso-lactide stream which have a high chemical and configurational purity, while at the same time the process can be operated in a flexible and cost-effective way. Further advantages of various embodiments of the method of the invention will become evident in the further specification.

### BRIEF DESCRIPTION OF THE DRAWINGS

The present invention will be discussed in more detail below. In the following, reference will be made to the following figures. The figures are for illustration purposes only; the invention is not limited thereto or thereby.

FIG. 1 illustrates a first embodiment of the process according to the invention, in which a feed comprising L-lactide, meso-lactide, water and lactic acid is subjected to a first separation step resulting in a crude lactide stream, the crude lactide stream is subjected to a first crystallisation step resulting in a purified L-lactide stream and a remnant stream comprising L-lactide and meso-lactide, subjecting the remnant stream to a second separation step resulting in a second purified L-lactide stream and a meso-lactide stream, and subjecting the meso-lactide stream to a second crystallisation step, resulting in a purified meso-lactide stream and a further remnant stream.

FIG. 2 illustrates a second embodiment of the process according to the invention, in which the first separation step consists of a two-step process.

FIG. 3 illustrates a third embodiment of the process according to the invention, in which a stream containing meso-lactide obtained from a first step of the first separation step is provided to the second separation step, and a stream containing L-lactide is obtained from the first separation step.

FIG. 4 illustrates a fourth embodiment of the process according to the invention, in which the first and the second separation step consist of a two-step process.

FIG. 5 illustrates a fifth embodiment of the process according to the invention, in which a stream high in L-lactide obtained from the second separation step is recycled to the first crystallisation step and a stream high in meso-lactide is forwarded to the second crystallisation step.

FIG. 6 illustrates a sixth embodiment of the process according to the invention, in which a stream containing L-lactide, meso-lactide, and contaminants obtained from the second distillation step is forwarded to a further separation step.

FIG. 7 illustrates a seventh embodiment of the process according to the invention, in which a lactic acid feed is subjected to a polycondensation step resulting in low-molecular weight polylactic acid, and the low-molecular weight polylactic acid obtained in the polycondensation step is subjected to a depolymerisation step, to obtain a lactide synthesis mixture.

The following is noted with respect to the figures:

The figures are intended to illustrate the invention. this invention is not limited thereto or thereby.

Embodiments of various figures can be combined unless they are mutually exclusive. The figures are flow sheets illustrating the process according to the invention. The figures do not present a reactor set up. For example, where a separation step is shown in a single step, it may be carried out on more than one reactor. Conversely, different steps may be carried out in the same unit. By the same token, the various lines are intended to show how components flow from one reaction step to the other. They do not represent real-life structures.

The figures do not always show all elements of the process according to the invention.

The figures do not show all purge streams or make-up streams that may be present in the practical performance of the process according to the invention although, as will be evident to the skilled person, purge streams and make-up streams may be necessary in practice to maintain stable operation.

The invention will be elucidated with reference to the figures, without being limited thereto or thereby.

### DETAILED DESCRIPTION OF THE INVENTION

### First Separation Step

In a first step, a feed comprising L-lactide, meso-lactide, water, lactic acid, and optional further components is subjected to a first separation step, the first separation step resulting in the formation of at least a stream comprising lactic acid and water and one or more crude lactide streams.

The feed provided to the first separation step may be indicated as lactide synthesis mixture. The lactide synthesis mixture generally comprises from 75 to 95 wt. % of L-lactide (based on the total weight of lactide in the stream), preferably 80 to 90 wt. %. The lactide synthesis mixture may comprise up to 5 wt. % of D-lactide (based on the total weight of lactide in the mixture), e.g. 0.01 to 5 wt. % (based on the total weight of lactide in the mixture), preferably at most 1 wt. %. The lactide synthesis mixture generally comprises from 1 to 25 wt. % of meso-lactide (based on the total weight of lactide in the mixture), preferably 2.5 to 10 wt. %. The total lactide content of the lactide synthesis mixture provided to the first separation step generally is in the range of 75 to 99.5 wt. %, calculated on the total weight of the mixture, in particular in the range of 85 to 98 wt. %. In addition to lactide, the mixture comprises lactic acid, generally in an amount of 0.01 to 10 wt. %, in particular in an amount of 0.5 to 5%. The feed further comprises water, generally in an amount of 0.01 to 4 wt. %, in particular in an amount of 0.1 to 2.5 wt% The feed composition, in particular the lactide content and composition may be determined using methods known in the art, including HPLC, e.g. using water/acetonitrile mixtures as eluent and UV-detection.

The lactide synthesis mixture may comprise further components, e.g., acid-containing impurities, such as lactoyl lactic acid, succinic acid and acetic acid. The acid-containing impurities may be present in the crude lactide stream in an amount such that the crude lactide stream has a free acid content of at least 20 meq/kg, in particular at least 50 meq/kg, and/or at most 150 meq/kg. As a maximum, the acid-containing impurities may be present in the crude lactide stream in an amount of at most 250 meq/kg. The free acid content as used herein can be determined by means of titration, e.g. using sodium methylate or potassium methylate in water-free methanol.

The first separation step is generally a distillation step, as distillation is an effective way to separate lactic acid and water on the one hand from lactide on the other hand. However, the first separation step may also be e.g. a pre-crystallization step. The first separation step may be carried out in a single unit, but it can also be carried out in a number of sequential units. For example, in one embodiment the first separation step is carried out in a two-step operation, wherein the feed is provided to a first separation unit, e.g., a distillation column, where a stream comprising water, lactic acid, and other volatile components is separated from a stream containing the majority of the lactide constituents. The stream containing the lactide constituents is provided at least in part to a second separation unit, generally a further distillation unit, where different lactide streams may be generated.

The first separation step generates one or more crude lactide streams. The determination of the number of crude lactide streams to be generated in this step depends in particular on the composition of the lactide fraction of the lactide synthesis mixture, in particular on the amount of meso-lactide as compared to L-lactide.

Specifically, as will be discussed below, a crude lactide stream comprising L-lactide and meso-lactide resulting from the first separation step will be subjected to a first crystallisation step, the first crystallisation step resulting in a first purified L-lactide stream and a remnant stream comprising L-lactide and meso-lactide. The composition of the crude lactide stream provided to this first crystallisation step is generally as follows:
L-lactide: between 80 and 98 wt. %, in particular between 85 and 95 wt. %, more preferably between 90 and 95 wt. %, calculated on the total weight of the crude lactide stream,
D-lactide: between 0 and 5 wt. %, calculated on the total weight of the crude lactide stream,
Meso-lactide: between 1 and 25 wt. % in particular between 2 and 12 wt. %, more preferable between 5 and 10 wt. %, calculated on the total weight of the crude lactide stream,
Lactic acid oligomers: between 0 and 7.5 wt. %, in particular at most 5 wt. %, calculated on the total weight of the crude lactide stream,
Other organic hydroxy acids below 2 wt. %, preferably below 1 wt. %, calculated on the total weight of the crude lactide stream,
Free acid level: between 0 and 5 wt. %, preferably at most 2 wt. %, more preferably at most 1.5 wt. %, even more preferably at most 1 wt. %, even more preferably at most 0.5 wt%, calculated on the total weight of the crude lactide stream.

Therefore, in one embodiment, the first separation step yields a crude lactide stream with a composition which is suitable for direct provision to the first crystallisation step, e.g., a composition as specified above. Furthermore, the total lactide content and the L-lactide content of the crude lactide stream are higher than the total lactide content and the L-lactide

content of the lactide synthesis mixture. In one embodiment, the first separation step yields a single crude lactide stream, containing both L-lactide and meso-lactide, which is then subjected to the first crystallisation step. This may be attractive in particular when the amount of meso-lactide is relatively limited as compared to the amount of L-lactide. It is preferred for the single crude lactide stream in this embodiment to have a composition as specified above.

In another embodiment, the first separation step generates a meso-lactide stream and a crude lactide stream comprising both L-lactide and meso-lactide. In this case, the crude lactide stream comprising both L-lactide and meso-lactide will then be subjected to the first crystallisation step. It is preferred for this crude lactide stream to have a composition as specified above for the feed to be provided to the first crystallisation step. The meso-lactide stream generally has a higher meso-lactide content than the crude lactide stream containing both L-lactide and meso-lactide that will be provided to the first crystallisation step. Typically the meso-lactide stream will contain lactic acid in an amount of from 0 to 10 wt. % and meso-lactide in an amount of 60 to 90 wt. %, calculated on the total weight of the meso-lactide stream. Furthermore, the meso-lactide stream may contain 0 to 30 wt. % L-lactide, calculated on the total weight of the meso-lactide stream.

This embodiment is advantageous because it ensures a high fraction of L-lactide and a low fraction of non-crystallising material in the crude lactide stream provided to the first crystallisation step, therewith preventing unnecessary unit load and improving separation efficiency and yield.

As indicated above, it is possible to carry out the first separation step in a two-step operation, wherein the lactide synthesis mixture is provided to a first separation unit, e.g., a distillation column, where a stream comprising water, lactic acid, and other volatile components is separated from a stream containing the majority of the lactide constituents. In one embodiment, part of the stream containing the majority of the lactide constituents is provided to a second separation unit, generally a further distillation unit, where different lactide streams are generated, while another part of the stream is provided directly to the first crystallisation step.

This embodiment is particularly attractive in cases where the lactide synthesis mixture contains relatively high amounts of water, lactic acid and/or meso-lactide, such as when a relatively high degree of racemization of the lactide occurs, or the throughput is relatively high.

The stream comprising water, lactic acid and other volatile components may also contain some meso-lactide. In an embodiment, the stream comprising water, lactic acid and other volatile components obtained from the first separation step, or from the first and/or second step of the first separation step, is provided to the second separation step. In this way, a higher meso-lactide yield is obtained.

In an embodiment, a stream comprising L-lactide is also withdrawn from the first separation step. Preferably, the stream comprising L-lactide contains at least 85 wt. % L-lactide, more preferably at least 90 wt. % L-lactide, even more preferable at least 95% wt. % L-lactide, as calculated based on the total weight of the stream. The stream comprising L-lactide generally contains some D-lactide. Preferably, the stream comprising L-lactide contains at most 15 wt. % D-lactide, more preferably at most 10 wt. %, even more preferable at most 5 wt. % D-lactide, as calculated based on the total weight of the stream. The composition of the stream comprising L-lactide withdrawn from the first separation step is suitable for providing the stream to a PLA manufacturing process.

Preferably, the crude lactide stream is condensed as part of the first separation step and obtained in liquid form. This is advantageous, since condensation of the crude lactide stream allows for the removal of highly volatile impurities, such as aldehydes and pyruvic acid, resulting in a purer crude lactide stream.

### First Crystallisation Step

In the process according to the invention a crude lactide stream comprising L-lactide and meso-lactide resulting from the first separation step is provided to a first crystallisation step. As indicated above, the stream to the first crystallisation step generally comprises:
L-lactide: between 80 and 98 wt. %, in particular between 85 and 95 wt. % and more preferably between 90 and 95 wt. %, calculated on the total weight of the crude lactide stream,
Meso-lactide: between 1 and 25 wt. % in particular between 2 and 12 wt. %, calculated on the total weight of the crude lactide stream,
further components as described above.

The first crystallisation step is carried out to generate a first purified L-lactide stream and a remnant stream comprising L-lactide and meso-lactide. The first crystallisation step is performed at a temperature of between 60 and 100° C., preferably between 70 and 90° C. Generally, a relatively low temperature is required when the L-lactide stream contains a relatively low concentration L-lactide and vice versa.

The aim of the first crystallisation step is to generate a high purity L-lactide fraction. The L-lactide fraction generated in this step generally contains at least 90 wt. % L-lactide, in particular at least 94 wt. % L-lactide, more in particular at least 96 wt. % L-lactide, or even at least 98 wt. % L-lactide, calculated on the total weight of the first purified L-lactide stream.

Preferably, the first purified L-lactide stream has a free acid level of <30 meq/kg, more preferably <15 meq/kg and even more preferably <5 meq/kg.

Generally the first purified L-lactide stream will contain a higher amount of L-lactide than the stream containing L-lactide obtained from the first separation step.

The remnant stream from the first crystallisation step comprises meso-lactide, which has not crystallised in the crystallisation step, and generally also L-lactide. The remnant stream may further comprise lactic acid and lactic acid oligomers, and other organic acids, such as other organic hydroxy acids. As the aim of this step is to manufacture high-purity L-lactide, the crystallisation conditions will be selected to ensure that this is the case. Accordingly, it is accepted that the remnant stream from the first crystallisation step still contains L-lactide.

In one embodiment, the remnant steam has the following composition: L-lactide in an amount of from 60 to 90 wt. %, meso-lactide in an amount of from 5 to 40 wt. %, lactic acid oligomers in an amount of from 0 to 10 wt. %, and other organic acids in an amount of from 0.5 to 2 wt. %, calculated on the total weight of the remnant stream.

It is possible to carry out the first crystallisation step in a two-step operation, wherein the crude lactide stream comprising L-lactide and meso-lactide resulting from the first separation step is provided to a pre-crystalliser where a pre-crystallisation takes place, resulting in a stream enriched in L-lactide and a remnant stream comprising meso-lactide and L-lactide. The stream enriched in L-lactide is then provided to a further crystalliser, resulting in the first purified L-lactide stream and the remnant stream comprising meso-lactide and L-lactide.

At least part of the remnant stream is subjected to the second separation step. Another part of the remnant stream from the pre-crystalliser and/or the first crystalliser can be recycled and mixed into the crude lactide feed for another crystallisation cycle, can be used as feed for another crystallisation batch, or can even be recycled further upstream, such as by mixing into the lactide synthesis mixture.

Furthermore, it is possible to recrystallise the L-lactide obtained from the first crystallisation step, in order to reach a desired yield and/or purity of the purified L-lactide stream.

Preferably, the purified L-lactide stream has a free acid level of <10 meq/kg, more preferably <5 meq/kg and even more preferably <3 meq/kg.

### Second Separation Step

At least part of the remnant stream from the first crystallisation step is provided to a second separation step, the second separation step resulting in at least a second L-lactide stream and a meso-lactide stream. The second separation step is generally a distillation step. The second separation step may be carried out in a single unit, but it can also be carried out in a number of sequential units. This will depend on the composition of the feed to the second separation step.

The remnant stream provided to the second separation step comprises meso-lactide, which has not crystallised in the first crystallisation step, and L-lactide. The remnant stream may further comprise lactic acid and lactic acid oligomers, and other organic acids, such as other organic hydroxy acids.

The second separation step yields a second L-lactide stream and a meso-lactide stream. The second L-lactide stream generated in this step is generally relatively pure, although not necessarily as pure as the purified L-lactide produced in the first crystallisation step. In one embodiment, the second L-lactide stream contains at least 80 wt. % L-lactide, in particular at least 90 wt. % L-lactide, more in particular at least 96 wt. % L-lactide, or event at least 98 wt. % L-lactide, calculated on the total weight of the second L-lactide stream. The second L-lactide stream may contain between 0 and 20 wt. % meso-lactide, calculated on the total weight of the second L-lactide stream. The second L-lactide stream may additionally also contain small quantities of lactic acid, D-lactide and other small lactic acid oligomers, organic acids and/or hydroxy acids other than lactic acid. Preferably, the second L-lactide stream contains <0.5 wt% lactic acid and <0.5 wt. % lactic acid oligomers, calculated on the total weight of the second L-lactide stream. Preferably, the second L-lactide stream contains <5 meq/kg lactic acid, calculated on the total weight of the second L-lactide stream.

The second L-lactide stream can be processed as desired, e.g., as discussed for the purified L-lactide resulting from the first crystallisation step. In one embodiment, the second L-lactide stream can be provided as feed to another crystallisation step, such as the first crystallisation step discussed above. This allows further purification of the L-lactide. In another embodiment, a third L-lactide stream is produced in the second separation step as a bottom stream which may serve as a feed to a crystallisation step. Since the primary function of such a crystallisation step is to recover L-lactide from the bottom stream, this results in an increased overall yield of L-lactide.

The second separation step also yields a meso-lactide stream. This stream will be provided to the second crystallisation step discussed below. The composition of the meso-lactide stream generated in the second separation step provided to the second crystallisation step is generally as follows: 80 to 98 wt. % meso-lactide, in particular 85 to 94 wt. %, 0 to 20 wt. % L-lactide, calculated on the total weight of the meso-lactide stream, traces of small high boiling lactic acid oligomers and organic acids other than lactic acid. The free acid content of the meso-lactide stream is preferably between 30 and 500 meq/kg.

The second separation step may also generate a lights fraction, containing, e.g., water, lactic acid, relatively small quantities of L-lactide and meso-lactide, and small organic acids such as acetic acid. Depending on the composition, it may be purged from the process or recycled in whole or in part to earlier steps, in particular to the first separation step, or to a lactide manufacturing step as will be discussed below.

The second separation step may also generate a bottoms fraction containing heavy-boiling contaminants, L-lactide, a relatively high amount of lactic acid oligomers with a degree of polymerisation more than 3 (i.e., oligomers comprising three or more lactic acid monomers). Depending on the composition it may be purged from the process or recycled in whole or in part to earlier steps, whether or not after intermediate separation.

In one embodiment, the bottoms fraction is subjected to a further crystallisation step, in which a further L-lactide-containing stream is generated, together with a further purge stream.

### Second Crystallisation Step

As indicated above, the meso-lactide stream generated in the second separation step is provided to the second crystallisation step. In the second crystallisation step meso-lactide is crystallised from the system, resulting in the generation of a purified meso-lactide stream.

The purified meso-lactide stream generated in this step generally contains at least 90 wt. % meso-lactide, in particular at least 94 wt. % meso-lactide, more in particular at least 96 wt. % meso-lactide, or even at least 98 wt. % meso-lactide, calculated on the weight of the purified meso-lactide stream. The purified meso-lactide stream may have a free acidity of less than 50 meq/kg, preferably less than 25 meq/kg, and in particular less than 10 meq/kg.

The second crystallisation step also generates a further remnant stream. The composition of the further remnant stream generally is as follows: between 60 and 90 wt. % meso-lactide, between 0 and 30 wt. % L-lactide, calculated on the weight of the further remnant stream. Depending on the crystallisation conditions, the further remnant stream may also comprise between 0 and 10 wt. % lactic acids and other organic acids. The further remnant stream may be purged, or can be recycled in full or in part to an earlier step, such as the first separation step, the first crystallisation step or the second separation step. Before recycling the further remnant stream to an earlier step it may optionally be subjected to an intermediate separation step.

The meso-lactide stream provided to the second crystallisation step may be crystallised by melt crystallisation. The meso-lactide stream may for instance be crystallised at a temperature of between 30 and 53° C., preferably at a temperature of between 35-45° C. The temperature chosen depends on the concentration meso-lactide of the meso-lactide stream. Generally, a relatively low temperature is required when the meso-lactide stream contains a relatively low concentration meso-lactide. Preferably, the second crystallisation step is only performed once.

The further remnant stream from the second crystallisation step can be processed as desired. Depending on its composition it can be purged from the system, but it can also be recycled to other steps, as a whole or in part, whether or not after intermediate separation. The further remnant stream may also be hydrolysed into a technical grade lactic acid with a low stereochemical purity.

### General Remarks on How the Different Steps May be Carried Out

As indicated above, the separation steps can be carried out in a single step, or in multiple steps, in a single unit or in multiple units. For instance, when the first separation step is performed in a two-step operation, both steps of the first separation step may be carried out in the same reactor or in different reactors. The first separation step may also be performed in the same reactor as the lactide synthesis mixture is generated in.

Distillation is often an attractive separation method, but depending on the compounds to be separated, other separation methods may also be applicable. Examples of such further separation methods include extraction, adsorption and absorption on ion exchange or carbon columns or functionalized resins.

Where the separation step is carried out in multiple steps, the steps may be based on the same separation mechanism, e.g., distillation, but it is also possible to combine steps which rely on different separation mechanisms, e.g., a combination of a distillation step and an extraction step.

Examples of suitable apparatus include distillation columns, divided wall columns, extraction columns, absorption columns, sublimation, etc.

Based on the guidance given above on the various fractions to be produced in the separation steps it is within the scope of the skilled person to select suitable separation steps, apparatus, and process conditions.

The method according to the invention comprises (at least) two crystallisation steps. Crystallisation steps can be carried out using conventional crystallisation techniques such as solvent crystallisation and melt crystallisation. Conventional apparatus may be used, e.g. layer crystallisation apparatuses like falling film and static crystallisers, but also suspension crystallisers like forced circulation crystallisers, scraped wall crystallisers, OSLO crystallisers and draft tube baffle crystallisers. Where layers crystallisation recovery proceeds by reheating to melt the crystals, suspension crystallisation typically requires a solid/liquid separation technique, like centrifuges, cyclones, filters or wash columns. Based on the guidance given above on the crystallisation steps it is within the scope of the skilled person to select suitable crystallisation steps, apparatus, and process conditions.

### Further Steps

As discussed above, the feed to the first separation step comprises L-lactide, meso-lactide, water, lactic acid, and optional further components. This feed may, e.g., be derived from a process for manufacturing lactide from lactic acid or depolymerization of PLA. As indicated above, lactides are commonly synthesized by oligomerizing lactic acid to form lactic acid oligomers via a polycondensation reaction. These oligomers are then depolymerized to form a lactide composition comprising L-lactide, D-lactide, and meso-lactide. The process according to the invention may also comprise the steps of subjecting lactic acid to a polycondensation step to manufacture lactic acid oligomers, followed by subjecting the lactic acid oligomers to a depolymerisation step to manufacture a crude lactide feed comprising L-lactide, meso-lactide, water, lactic acid, and optional further components.

In the first step a low-molecular weight poly(lactic acid) is formed by condensation polymerisation of lactic acid. The lactic acid is often obtained from a biological process and generally has a high optical purity. Depending on the source it may contain at least 90% of L-lactic acid, in particular at least 95% L-lactic acid, more in particular at least 98 wt. %. The condensation polymerisation may be carried out as is known in the art. It generally involves subjecting lactic acid to sub-atmospheric pressure, e.g. 50-500 mbar at elevated temperatures, e.g., 100-200°C, to induce polymerisation by removal of water. The average degree of polymerisation obtained is generally between 5 and 20. The low-molecular weight polylactic acid obtained in the first step is then subjected to a depolymerisation step, to convert the low molecular weight polylactic acid to lactide. Depolymerisation is also known in the art. It is generally carried out in the presence of a catalyst. Metal-containing catalysts are often used, in particular catalysts based on tin, zinc, aluminium, lead, antimony, lead, calcium, and magnesium, e.g., in the form of halide salts or salts of organic acids, such as fatty acids. Tin (II) bis(2-ethyl hexanoate) is often used commercially. Typical concentration of lactide synthesis catalysts may be 20-2000 ppm. Reaction conditions include a temperature of 160 to 260°C, a pressure of 5 to 100 mbar, and a residence time of 10 minutes to 8 hours.

Streams containing lactic acid generated in the process according to the invention, e.g., in the first separation step or in the second separation step may be recycled to the polycondensation step. If so desired, streams containing meso-lactide may be forwarded to a downstream crystallisation step. For example, a meso-lactide containing stream resulting from the first separation step which is rich in meso-lactide and contains a relatively low amount of L-lactide may be forwarded to the second separation step, bypassing the first crystallisation step. Also streams high in L-lactide may be recycled, for instance to further increase the L-lactide content of the stream. The second L-lactide stream may for example be recycled to the first crystallisation step for this purpose. In addition to this, lactide comprising streams originating from external sources such as PLA production may be fed to the first crystallisation step, the second separation step and/or the second crystallisation step.

The L-lactide and meso-lactide generated by the various steps in the process according to the invention can be processed as desired. In one embodiment at least a portion of the L-lactide resulting from the first crystallisation step or at least a portion of the meso-lactide resulting from the second crystallisation step, or the combination of at least a portion of the L-lactide resulting from the first crystallisation step and at least a portion of the meso-lactide resulting from the second crystallisation step are provided to a polymerisation step to form a polymer comprising lactide units. Depending on whether or not further monomers are added, the polymer may be a polylactide homopolymer or a polylactide co-polymer. In the latter case further monomers which are polymerisable with the lactide monomers. Examples of further monomers include glycolide, and epsilon caprolactone, resulting in the formation of poly(lactide-co-glycolide), poly(lactide-co-epsilon-caprolactone), and poly(lactide-co-glycolide-co-epsilon-caprolactone).

The L-lactide and meso-lactide produced in the process according to the invention may also be used for production of high purity lactic acid. It is also possible to apply them in other applications, e.g., in coatings, sealants, adhesives, resins and hot melts or production of esters.

The present application is drafted for a method for processing a lactide feed comprising L-lactide as the predominant lactide isomer. It will however be clear to the skilled person that the same process as described in the present application applies to a lactide feed comprising D-lactide as the predominant lactide isomer, and that where the term “L-lactide” is used in the present application this could be replaced by “D-lactide” and vice versa. It will be clear to the skilled person that such a process of processing a lactide feed comprising D-lactide as the predominant isomer also falls within the scope of the present application.

As will be evident to the skilled person, different embodiments of the present invention can be combined unless they are mutually exclusive. When amounts, concentrations, dimensions and other parameters are expressed in the form of a range, a preferable range, an upper limit value, a lower limit value or preferable upper and limit values, it should be understood that any ranges obtainable by combining any upper limit or preferable value with any lower limit or preferable value are also specifically disclosed, irrespective of whether the obtained ranges are clearly mentioned in the context.

It is furthermore noted that headings are provided for the convenience of the reader, but are not limiting the invention. Furthermore, the terms “stream”, “feed” and “fraction” may be used interchangeably herein, and streams can be withdrawn or provided to a subsequent step in whole or in part, also when this is not specified.

### Illustrative Embodiments

The present invention is illustrated by the following figures, without being limited thereto or thereby.

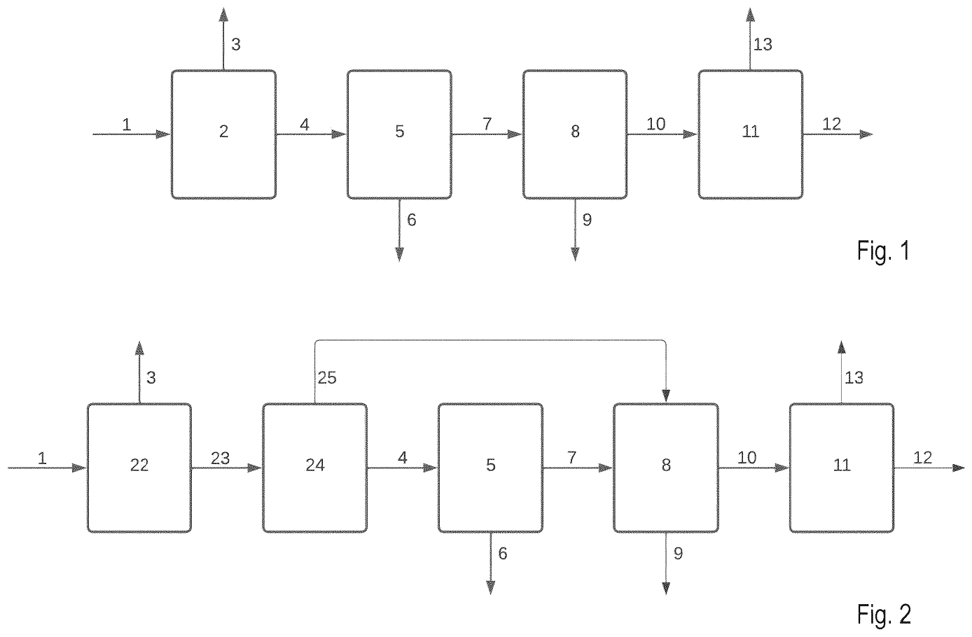

FIG. 1 provides a general illustration of the process of the invention. In FIG. 1, a feed comprising meso-lactide, L-lactide, water, lactic acid, and optional further components is provided through line (1) to separation step (2). Separation step (2) generates a stream (3) comprising lactic acid and water, and a crude lactide stream (4) comprising L-lactide and meso-lactide. Stream (4) comprising L-lactide and meso-lactide is provided to a first crystallisation step (5). Crystallisation step (5), which may consist of one or more stages, generates a high-purity L-lactide, which is withdrawn through line (6). The L-lactide stream generally has an L-lactide content of at least 90%, in particular at least 95%, more in particular at least 98%, in some embodiments at least 99%, calculated on total lactide. Crystallisation step (5) also generates a remnant stream (7), which comprises L-lactide and meso-lactide. Remnant steam (7) is provided to second separation step (8). Second separation step (8) results in a second L-lactide stream (9), and a meso-lactide stream (10). Meso-lactide stream (10) is provided to second crystallisation step (11), where meso-lactide is crystallised in purified form, and withdrawn from the process through line (12). A further remnant stream is withdrawn through line (13).

FIG. 2 illustrates some aspects of the present invention in more detail. In FIG. 2, the separation step consists of two distillation steps (22) and (24). In distillation step (22) lactic acid and water are separated off, and are withdrawn through line (3). The effluent from the first distillation step (22) has a high lactide content. It is withdrawn through line (23), and provided to the second distillation step (24). Second distillation step (24) generates a lactide stream which is rich in meso-lactide, which is withdrawn through line (25). Second distillation step (24) also generates a crude lactide stream (4), comprising L-lactide and meso-lactide, which is provided to first crystallisation step (5). Thus, in this embodiment, at least some meso-lactide is already separated from the lactide fraction before the first crystallisation step (5). This is advantageous when the feed has a relatively high meso-lactide content, because in that case removal of some meso-lactide before the first crystallisation step results in a reduced load on the first crystallisation step (5). The lactide stream which is rich in meso-lactide which is withdrawn through line (25) can be provided to second separation step (8), where it is subjected to further separation. This embodiment is shown in FIG. 2. This embodiment is advantageous, because in this way the amount of meso-lactide in the crude lactide stream (4) comprising L-lactide and meso-lactide which is provided to the first crystallisation step (5) is reduced. Therefore, the first crystallisation step (5) can be performed more efficiently. Furthermore, L-lactide present in the lactide stream which is withdrawn through line (25) can be recovered when the stream is forwarded to the second separation step (8). This contributes to a higher overall yield of L-lactide.

Alternatively, and depending on its composition, it is also possible to provide the lactide stream which is rich in meso-lactide which is withdrawn through line (25) directly to second crystallisation step (11). This embodiment, which is not shown in FIG. (2) is particularly attractive when the meso-lactide stream (25) withdrawn from the second distillation column (24) has a relatively high meso-lactide content, and a relatively low content of, in particular, L-lactide. In this way, the first crystallisation step is performed more efficiently, resulting in a higher overall yield of meso-lactide.

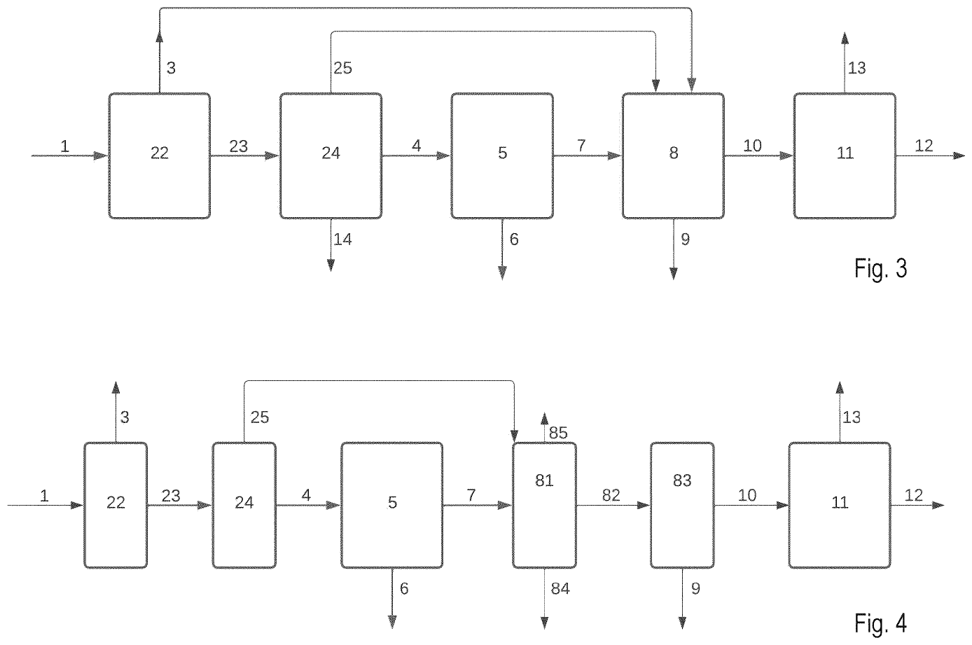

FIG. 3 illustrates a variant of the embodiment shown in FIG. 2. In addition to lactic acid and water, the stream withdrawn through line (3) from the first distillation step (22) of the first separation step still contains meso-lactide. This stream (3) is provided to the second separation step (8) where it is subjected to further separation. In this way, the meso-lactide yield of the process is increased. Furthermore, stream (14) containing L-lactide is withdrawn from the second distillation column (24). Stream (14) has a relatively high L-lactide content, but generally also contains some impurities such as some D-lactide. However, the composition of stream (14) is generally suitable to be provided to a PLA manufacturing process. Withdrawing stream (14) from the second separation step results in a smaller volume of stream (4) being provided to the first crystallisation step (5). Therefore, smaller equipment can be used for the first and second crystallisation step and the second separation step, resulting in a cost reduction of the process.

FIG. 4 illustrates an embodiment which is another variation on the embodiment of FIG. 2. In the embodiment of FIG. 4, second separation step (8) consists of two distillation steps (81) and (83). Remnant stream (7) originating in first crystallisation step (5) is provided to first distillation step (81). In the illustrated embodiment the same applies for the steam rich in meso-lactide (25). However, this stream is optional only, as discussed above. In distillation step (81) a light fraction, containing water, lactic acid and optional other volatile components is separated off through line (85). Depending on its composition, it may be recycled to other process steps, whether or not after intermediate purification, or purged from the process. A fraction containing heavy components, e.g., lactide oligomers generated during the process is withdrawn through line (84). Depending on its composition, it may be recycled to other process steps, whether or not after intermediate purification, or purged from the process. The first distillation step (81) also generates a stream with a high lactide content. This stream is withdrawn through line (82), and provided to second distillation step (83). Second distillation step (83) generates a second L-lactide feed, which is withdrawn through line (9). A feed rich in meso-lactide is withdrawn through line (10) and provided to second crystallisation step (11). The second distillation step (83) may also generate a bottoms fraction (not shown) which is withdrawn from the step, and provided, e.g., to first distillation step (81) or otherwise disposed of. By the same token, the second distillation step (83) may also generate a top fraction (not shown) which is withdrawn from the step, and provided, e.g., to first distillation step (81) or otherwise disposed of. The advantage of carrying out the second separation step in two sequential steps, in particular as two sequential distillation steps, is that both the L-lactide stream generated in this step and the stream provided to the second crystallisation step can be relatively pure, due to the preceding separation of a light fraction and a heavy fraction in the first distillation step. Thus, the meso-lactide stream can be processed efficiently in the second crystallisation step, and a better quality L-lactide is obtained compared to an embodiment in which the second separation step consists of one single step.

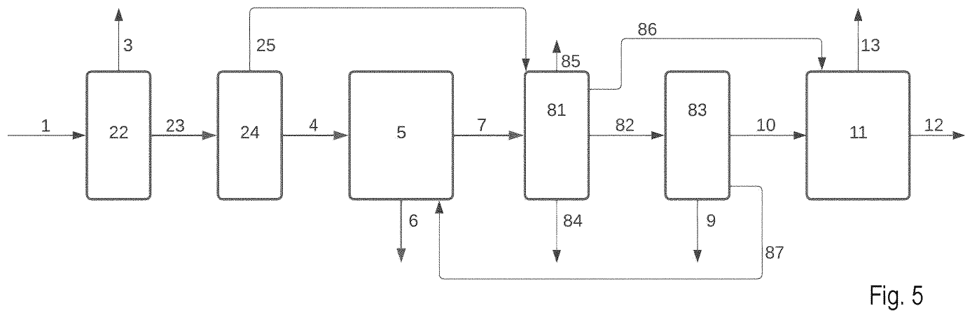

FIG. 5 shows a variation on FIG. 4. In the embodiment of FIG. 5, the first distillation step (81) of the second separation step (2) also generates a meso-lactide stream (86), which is provided directly to second crystallisation step (11) and bypasses second distillation step (83). Whether or not this embodiment is attractive depends on how the first distillation step (81) can be operated. In the embodiment illustrated in FIG. 5 the second distillation step (83) also yields a stream (87) which is relatively high in L-lactide, but still contains some contaminants. As compared to L-lactide stream (9), stream (87) has a higher contaminant content. It therefore benefits from further purification, and, in the embodiment illustrated in FIG. 5, is recycled to the first crystallisation step (5). By providing meso-lactide stream (86) directly to the second crystallisation step (11), the overall yield of meso-lactide increased and the contamination of stream (87) is reduced.

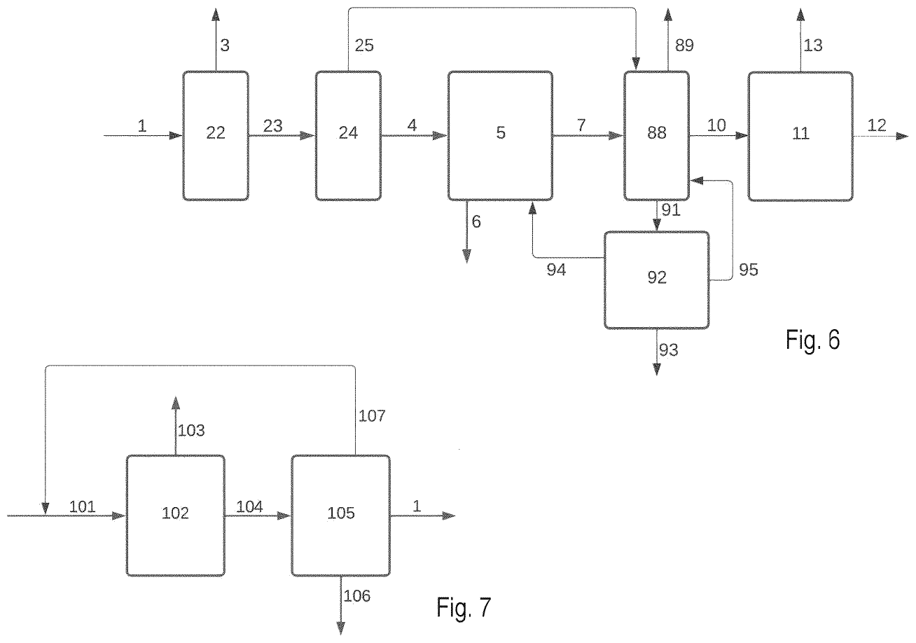

FIG. 6 shows a further embodiment in which the second separation step is carried out in two steps, namely distillation steps (88) and (92). In the embodiment illustrated in this figure, remnant stream (7) originating in first crystallisation step (5) is provided to first distillation step (88). In the illustrated embodiment the same applies for the steam rich in meso-lactide (25). Distillation step (88) generates a stream rich in meso-lactide which is provided to second crystallisation step (11) through line (10). In the illustrated embodiment, distillation step (88) generates a lights stream (89), which may be withdrawn from the process or, e.g., recycled to a lactide manufacturing step not shown. Distillation step (88) also generates a stream containing a substantial amount of L-lactide, some meso-lactide, and some contaminants. This stream is passed through line (91) to a second distillation step (92). In step (92) a purified L-lactide is separated, and withdrawn through line (93). In the embodiment illustrated in the figure, a fraction containing meso-lactide is withdrawn through line (95), and recycled back to the first distillation step (88), with the aim to put the meso-lactide in this fraction towards the second crystallisation step (11). In the embodiment illustrated in the figure, the second distillation step (92) also yields a stream (94) which is relatively high in L-lactide, but still contains some contaminants. As compared to L-lactide

stream (93), stream (94) has a higher contaminant content. It therefore benefits from further purification, and, in the embodiment illustrated in FIG. 6, is recycled to the first crystallisation step (5). The L-lactide stream (93) may be sent to a crystallisation step, such as the first crystallisation step (5) or the second crystallisation step (not shown). This embodiment is advantageous, because it results in a higher yield of meso-lactide and L-lactide, and to meso-lactide and L-lactide with a higher purity.

FIG. 7 illustrates one embodiment of manufacturing the feed (1) as it may be provided to the method of the invention. In the embodiment of FIG. 7, lactic acid is provided through line (101) to a polycondensation step (102), where lactic acid oligomers are produced with removal of water through line (103). The lactic acid oligomers are transported to depolymerisation step (105) through line (104). In the depolymerisation step (105), lactide is formed, which then enters the process through line (1) where it is provided to separation step (2). As indicated above, the various boxes in the figures are not intended to represent separate process steps. For example, it is possible to carry out a depolymerisation step in a step which also performs the first separation step. In such a case a single step would encompass depolymerisation step (105) and separation step (2). In such a case there would not be an explicit feed provision line (1). Depolymerisation step (105) may generate a bottoms steam, shown in FIG. 7 as bottoms stream (106). This stream may contain lactic acid oligomers and contaminant compounds. It may be processed as desired. In one embodiment it may be hydrolysed to form technical grade lactic acid. Depolymerisation step (105) may also generate a lights steam, shown in FIG. 7 as lights stream (107). Lights stream (107) will generally contain lactic acid. Depending on the conditions in depolymerisation step (105), it may also comprise some meso-lactide. Lights stream (107) can be processed as desired. In one embodiment it is provided to polycondensation step (102). It is also possible to provide it to lactic acid feed (101) as is illustrated in FIG. 7, with or without having been subjected to a hydrolysis step (not shown) to convert lactide or other lactic acid oligomers in the stream to lactic acid.

1. A method for processing a lactide feed, comprising the steps of
(a) subjecting a feed comprising L-lactide, meso-lactide, water, lactic acid, and optional further components to a first separation step, the first separation step resulting in the formation of a stream comprising lactic acid and water and one or more crude lactide streams,
(b) subjecting at least a portion of a crude lactide stream comprising L-lactide and meso-lactide resulting from the first separation step to a first crystallisation step, the first crystallisation step resulting in a purified L-lactide stream and a remnant stream comprising L-lactide and meso-lactide,
(c) subjecting the remnant stream comprising L-lactide and meso-lactide resulting from the first crystallisation step to a second separation step, the second separation step resulting in at least a second L-lactide stream and a meso-lactide stream,
(d) subjecting at least a portion of the meso-lactide stream resulting from the second separation step to a second crystallisation step, resulting in the formation of a purified meso-lactide stream and a further remnant stream.

2. The method according to claim 1, wherein the first and/or second separation step comprises a distillation step.

3. The method according to claim 1, wherein the first and/or second separation step is a multi-step process.

4. The method according to claim 1, wherein the first and/or second separation step is a two-step process.

5. The method according to claim 1, wherein the first and/or second crystallisation step is a multi-step process.

6. The method according to claim 1, wherein the first and/or second crystallisation step is a two-step process.

7. The method according to claim 1, wherein a stream rich in meso-lactide obtained from the first separation step is provided to the second separation step or the second crystallisation step.

8. The method according to claim 1, wherein a lactide stream comprising L-lactide is condensed as part of the first separation step.

9. The method according to claim 1, wherein a stream comprising lactide obtained from the second separation step is provided to the first crystallisation step.

10. The method according to claim 1, wherein the further remnant stream obtained from the second crystallisation step is provided to the first separation step, the first crystallisation step and/or the second separation step.

11. The method according to claim 1, wherein the first separation step yields a single lactide stream, or
wherein the first separation step yields a meso-lactide stream and a crude lactide stream comprising L-lactide and meso-lactide.

12. The method according to claim 1, wherein a meso-lactide stream resulting from the first separation step is provided to the second separation step or the second crystallisation step.

13. The method according to claim 1, wherein the second L-lactide stream is provided to the first crystallisation step.

14. The method according to claim 1, wherein the feed comprising L-lactide, meso-lactide, water, lactic acid, and optional further components comprises from 75 to 95 wt. % L-lactide, based on the total weight of lactide.

15. The method according to claim 1, wherein the feed comprising L-lactide, meso-lactide, water, lactic acid, and optional further components comprises from 75 to 99.5 wt. % total lactide, based on the total weight of the feed.

16. The method according to claim 1, wherein the feed comprising L-lactide, meso-lactide, water, lactic acid, and optional further components has a free acid content of at least 20 meq/kg, and/or at most 150 meq/kg.

17. The method according to claim 1, wherein the first separation step is preceded by
a step of subjecting lactic acid to a polycondensation step to manufacture lactic acid oligomers, and
subjecting the lactic acid oligomers obtained in the polycondensation step to a depolymerisation step to manufacture a feed comprising L-lactide, meso-lactide, water, lactic acid, and optional further components.

18. The method according to claim 17, wherein the stream comprising lactic acid resulting from the first separation step is provided to the step of subjecting lactic acid to a polycondensation step.

19. The method according to claim 17, wherein the depolymerisation step and the first separation step are performed in the same reactor.
